(19) United States
(12) Patent Application Publication
(10) Pub. No.: US 2025/0186185 A1
(43) Pub. Date: Jun. 12, 2025

(54) IPS PREPROSTHETIC DEVICE

(71) Applicant: KLS-Martin L.P., Jacksonville, FL (US)

(72) Inventors: Philipp PIZZITOLA, Rottweil (DE); Kirsten STUCK, Jacksonville, FL (US); Nathan JEFFERS, Jacksonville Beach, FL (US); Bennie Wayne GLADDISH, Jr., Odessa, FL (US); Justin LYONS, Ponte Vedra, FL (US)

(21) Appl. No.: 18/971,343

(22) Filed: Dec. 6, 2024

Related U.S. Application Data
(60) Provisional application No. 63/607,183, filed on Dec. 7, 2023.

(51) Int. Cl. A61F 2/00; A61F 2/30

(52) U.S. Cl. CPC A61F 2/00; A61F 2002/30329; A61F 2002/30535

(57) ABSTRACT

An implant operably coupleable to a bone of a patient may include at least one securing portion which may be configured to receive fastening members to operably couple the implant to the bone, a support structure which may be conformal to contours of the bone, a plurality of pillars which may extend from, and be formed integrally with, the support structure, and a plurality of abutments which may be disposed at the plurality of pillars. The plurality of abutments may be operably coupleable to a prosthesis.

### TECHNICAL FIELD

Example embodiments generally relate to preprosthetic implants for attaching to bone, and in particular, relate to a preprosthetic implant that provides support for multi-unit prostheses.

### BACKGROUND

More than 34 million Americans are missing all of their teeth in at least their upper or lower jaw. Jaw implants are well known and typically include substantially plate-shaped configuration which can be adapted to the contour of the bone. Typical implants may include a body portion, which may be the portion of the implant that may be buried in the patient's bone and may be usually threaded for fixation. An abutment is the portion that may screw into the body and may typically be available in many shapes and sizes. Finally, the crown or bridge portion represents the actual replacement teeth and may be affixed in some manner to the abutment using whatever method that may be preferred by the dental surgeon.

Traditional dental implants may be a viable option for patients with adequate bone and soft tissue. However, when a patient is exhibiting severe jaw atrophy or bone loss, traditional implants may not be an option. Thus, it may be desirable to design a preprosthetic implant to provide a more individualized approach to dental rehabilitation for circumstances in which there may be insufficient bone stock available for conventional dental implants.

### BRIEF SUMMARY OF SOME EXAMPLES

In an example embodiment, an implant operably coupleable to a bone of a patient may be provided. The implant may include at least one securing portion which may be configured to receive fastening members to operably couple the implant to the bone, a support structure which may be conformal to contours of the bone, a plurality of pillars which may extend from, and be formed integrally with, the support structure, and a plurality of abutments which may be disposed at the plurality of pillars. The plurality of abutments may be operably coupleable to a prosthesis.

Some example embodiments may provide for method of employing an implant on a patient. The method may include the steps of making a scan of a bone of the patient, making a 3D image of the bone of the patient using the scan, making a design of the implant to conform to the bone using the 3D image, and producing the implant using the design. The method may optionally further include the steps of operably coupling the implant to the patient, and operably coupling the prosthesis to the implant. The prosthesis may be operably coupled to the implant via a plurality of abutments disposed at a plurality of pillars of the implant.

### BRIEF DESCRIPTION OF THE SEVERAL VIEWS OF THE DRAWING(S)

Having thus described some example embodiments in general terms, reference will now be made to the accompanying drawings, which are not necessarily drawn to scale, and wherein:

FIG. 1 illustrates a front view of an implant for the mandible in accordance with an example embodiment;

FIG. 2 illustrates a front view of an implant for the maxilla in accordance with an example embodiment;

FIG. 3 illustrates a perspective view of the implant for the maxilla in accordance with an example embodiment;

FIG. 4 illustrates a close-up front view of the implant for the maxilla having milled abutments in accordance with an example embodiment;

FIG. 5 illustrates a close-up perspective view of the milled abutment of the implant in accordance with an example embodiment;

FIG. 6 illustrates a close-up front view of the implant for the maxilla having removable abutments in accordance with an example embodiment;

FIG. 7 illustrates a front view of various sizes of removable abutments for the implant in accordance with an example embodiment;

FIG. 8 illustrates a close-up front view of the implant for the maxilla in accordance with an example embodiment;

FIG. 9 illustrates a perspective view of a portion of the implant in accordance with an example embodiment;

FIG. 10 illustrates a left side view of the implant in accordance with an example embodiment;

FIG. 11 illustrates a perspective view of the implant for the maxilla in accordance with an example embodiment;

FIG. 12 illustrates a flow chart of a method of employing an implant on a patient in accordance with an example embodiment;

FIG. 13 illustrates a close-up perspective view of a portion of the implant in accordance with an example embodiment;

FIG. 14 illustrates a close-up view of a portion of the support structure in accordance with an example embodiment;

FIG. 15 illustrates a close-up bottom view of a portion of the implant in accordance with an example embodiment;

FIG. 16 illustrates a close-up perspective view of a portion of the implant in accordance with an example embodiment;

FIG. 17 illustrates separate views of a 2D mesh pattern having various layouts in accordance with an example embodiment; and

FIG. 18 illustrates a close-up perspective view of a portion of the implant in accordance with an example embodiment.

### DETAILED DESCRIPTION

Some example embodiments now will be described more fully hereinafter with reference to the accompanying drawings, in which some, but not all example embodiments are shown. Indeed, the examples described and pictured herein should not be construed as being limiting as to the scope, applicability or configuration of the present disclosure. Rather, these example embodiments are provided so that this disclosure will satisfy applicable legal requirements. Like reference numerals refer to like elements throughout. Furthermore, as used herein, the term “or” is to be interpreted as a logical operator that results in true whenever one or more of its operands are true. As used herein, operable coupling should be understood to relate to direct or indirect connection that, in either case, enables functional interconnection of components that are operably coupled to each other.

Additionally, as used herein, terminology such as “about,” “approximately” and “substantially,” when used to refer to variability of parameters, should be understood to be definite approximations that account for variations in measurements that cannot be, or as one of skill in the art would appreciate, normally are not, measured precisely. Thus, for example, a parameter that is “about,” “approximately” or “substantially” a given value or a given characteristic should be understood to be sufficiently close to the given value or given characteristic such that performance of the object or product to which the parameter applies, from the perspective of one with ordinary skill in the art, is the same as though the object or product had precisely the given value or characteristic.

The preprosthetic implant of the present disclosure may be designed for patients for whom a clinical necessity may exist. These may include patients with acquired malformations caused by trauma or atrophy but also those with congenital malformations who, as they get older, may lose their teeth and may exhibit poor bone and soft tissue quality. When attempting to provide these patients with a conventional dental implant restoration, a point may be reached where they cannot be treated adequately, due to bone loss, which may therefore result in the need for an alternative solution.

With the preprosthetic implant of the present disclosure, a surgeon can operate independently of the bone volume because the bone does not necessarily have to be situated where conventional implants would be placed. Advantageously, the existing bone structure can be analyzed, and a specific implant design can be created, leading to the creation of an implant that may conform to the patient's bone to create a prosthetic solution.

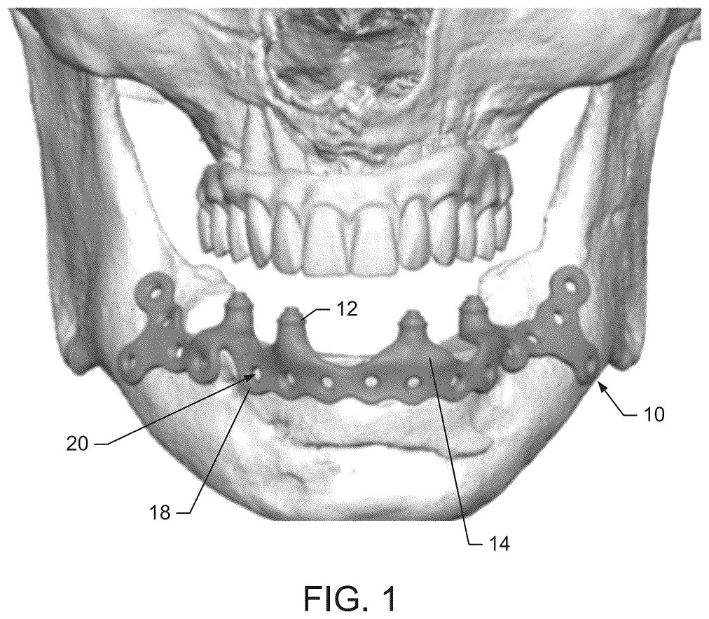

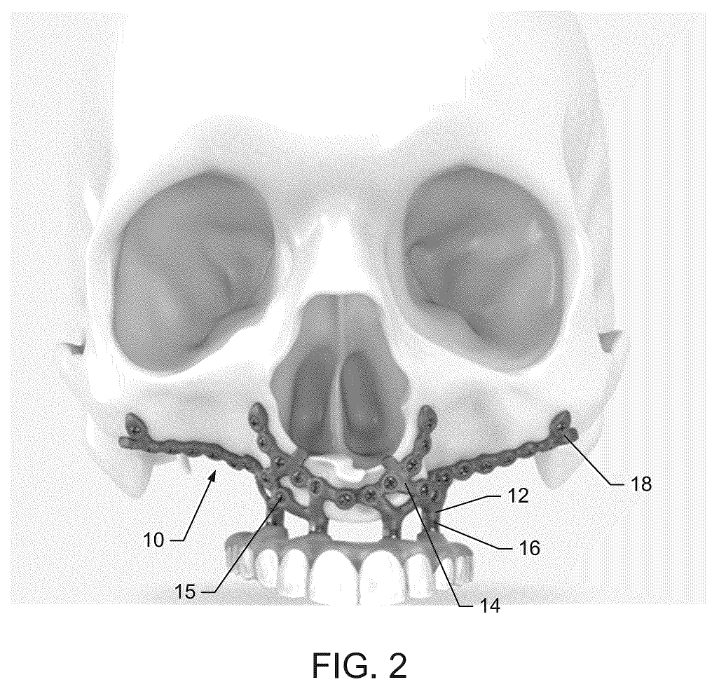

Referring now to FIGS. 1-3, the preprosthetic implant 10 of the present disclosure can be seen in various embodiments. As shown in FIG. 1, the implant 10 may conform to the contours of, and be operably coupled to, a patient's lower jaw, or mandible. Implant 10 may include a plurality of pillars 12, in this case four, which may be formed integrally with a support structure 14. In one embodiment, pillars 12 may be hollow-cylindrical and may have different heights and angles. While implant 10 may be disposed beneath the oral mucosa and the periosteum, the pillars 12 may project therefrom into the oral cavity. In some cases, each pillar 12 may be designed to conform to the patient's bone anatomy. In an example embodiment, each pillar can also be angulated relative to the occlusal plane. Extending from each pillar 12 may be a corresponding abutment 16. Each abutment 16 may be operably coupled to the replacement tooth or denture. Implant 10 may be configured for holding a prosthesis (not shown) via one or more abutments 16. This will be discussed below in further detail with regard to FIG. 2.

Support structure 14 may also include a plurality of securing portions 18, which may extend outward from support structure 14 and may be arranged in a grid-like structure. The grid-like structure of securing portions 18 may vary and, advantageously, may be adapted to the bone structure of each patient. The shape of support structure 14 including each securing portion 18 may be adapted to the patient's bone structure upon which implant 10 may be placed. In some cases, securing portions 18 may include a plurality of annular portions 20. In an example embodiment, securing portions 18 may serve to secure implant 10 to existing bone structures.

Securing portions 18 may be affixed to the patient's bones by fastening members 15, which may project through corresponding annular portions 20, to secure implant 10 to the patient's bone. In some cases, the fastening members 15 may be screws. The annular portions 20 that include the securing portions 18 may be angled to provide optimal intraoperative fixation. The embodiment shown in FIG. 1 can be applied to both partially toothed or toothless mandibles so as to compensate for missing teeth by means of a dental prosthesis which may be supported by implant 10.

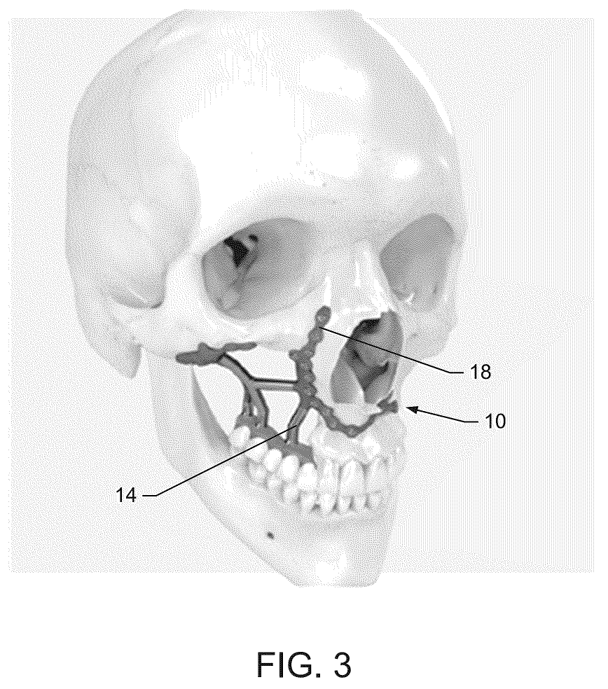

As shown in FIGS. 2 and 3, the implant 10 may be operably coupled to the patient's upper jaw, or maxilla. FIGS. 2 and 3 illustrate how support structure 14 and securing portions 18 may adapt to the contour of the patient's bone. This adaptation may be achieved by various means including using a scan of the patient's oral template and proprietary software to create a 3D image of the patient's jaw in order to design implant 10 that may be customized to conform to that patient's specific contours. A 3D printer may be used to print a mold which may then be annealed and milled until a final implant 10 is produced.

Also seen in FIGS. 2 and 3, a plurality of (in this case four and three) downwardly-directed pillars 12 may extend from support structure 14 of implant 10. In one embodiment, each pillar 12 may be of a different height in order to adjust to the bone structure of the patient. Thus, it is within the scope of the present disclosure to provide an implant 10 that may have pillars 12 of varying angles and heights.

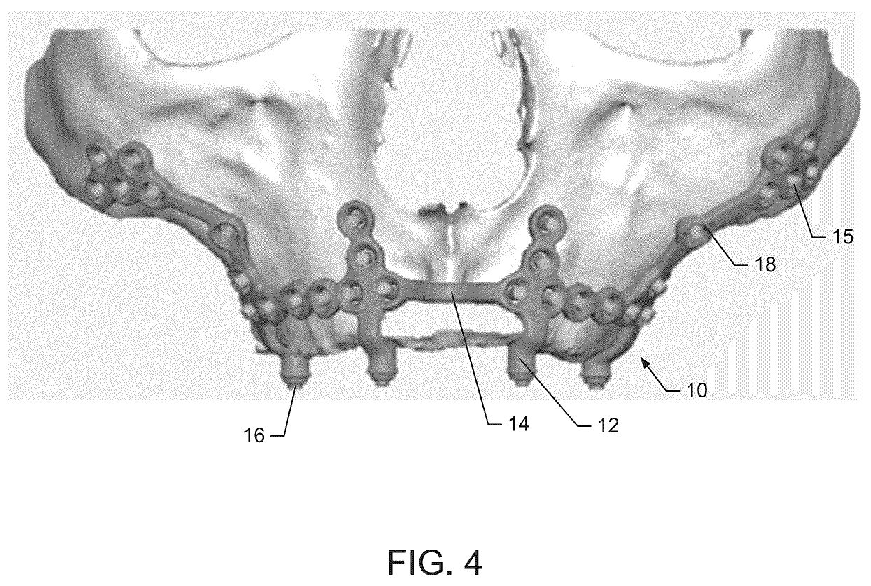

FIG. 4 illustrates one embodiment of the implant 10 of the present disclosure, in which each abutment 16 (in this case four) may be pre-milled into a corresponding pillar 12 of the support structure 14. Prosthesis milling specifications are available to accommodate a number of connection methods. These may include, but are not limited to, a direct M1.4 screw into MUA 16; a temporary coping fixated within the screw, and luted with light cured acrylic; and a Ti Base fixated with the screw, and luted with acrylic lightly cured. It is within the present disclosure to include other types of prosthesis milling specifications other than those mentioned herein.

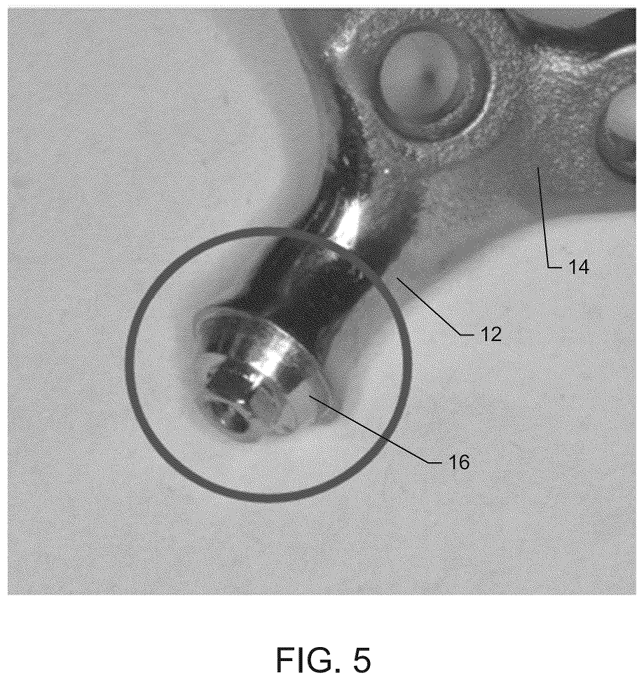

FIG. 5 shows a close-up view of an exemplary pre-milled abutment 16 in accordance with an example embodiment. In this regard, abutment 16 may be pre-milled and may form an integral part of pillar 12 of support structure 14 of implant 10.

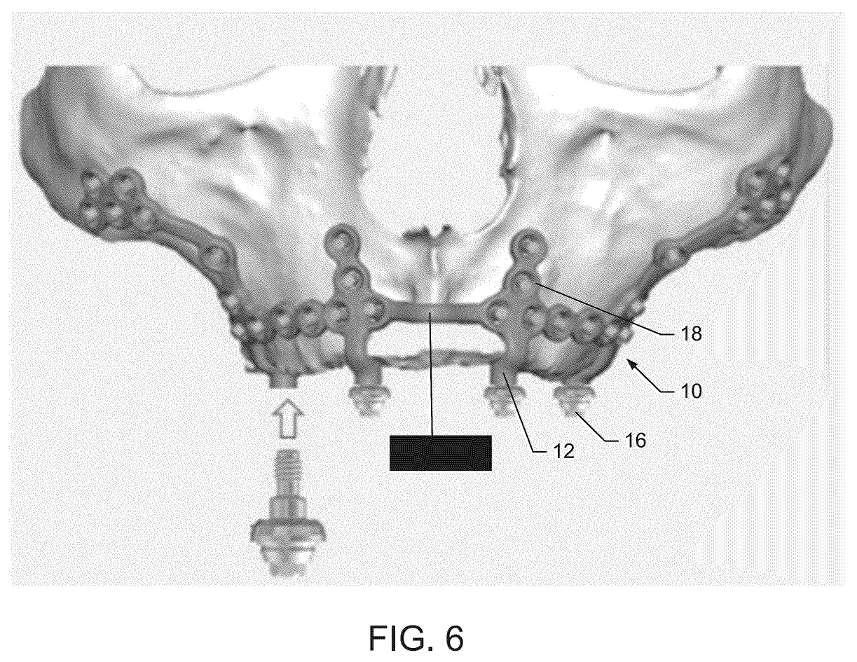

FIG. 6 illustrates another embodiment of the implant 10 of the present disclosure. In the embodiment of FIG. 6, the multi-unit abutment (MUA) 16 may not be milled with, or integral with, the support structure 14 or pillars 12, but instead may form a separate unit. In this regard, the abutments 16 may be removably operably coupled to the pillars 12. In other words, implant 10 may not be limited to pre-formed abutments 16, which may thus give the surgeon flexibility to request abutments 16 of different sizes and dimensions for each patient. As such, implant 10 may be milled to the implant level, rather than the abutment level. This may avoid a “fixed system” which occurs when the preprosthetic implant may be milled to the level of the MUA 16.

The advantage of milling implant 10 to the implant level is that predicting soft-tissue interaction to a subperiosteal implant may be nearly impossible. Machining preprosthetic implants to the implant level may allow the user to add on additional length abutments 16 if needed to emerge through unexpected soft tissue growth. Another advantage of such an approach is if a thread on an abutment 16 strips or becomes otherwise inoperable, the abutment 16 can simply be swapped out for another, rather than having to remove and replace the entire support structure 14.

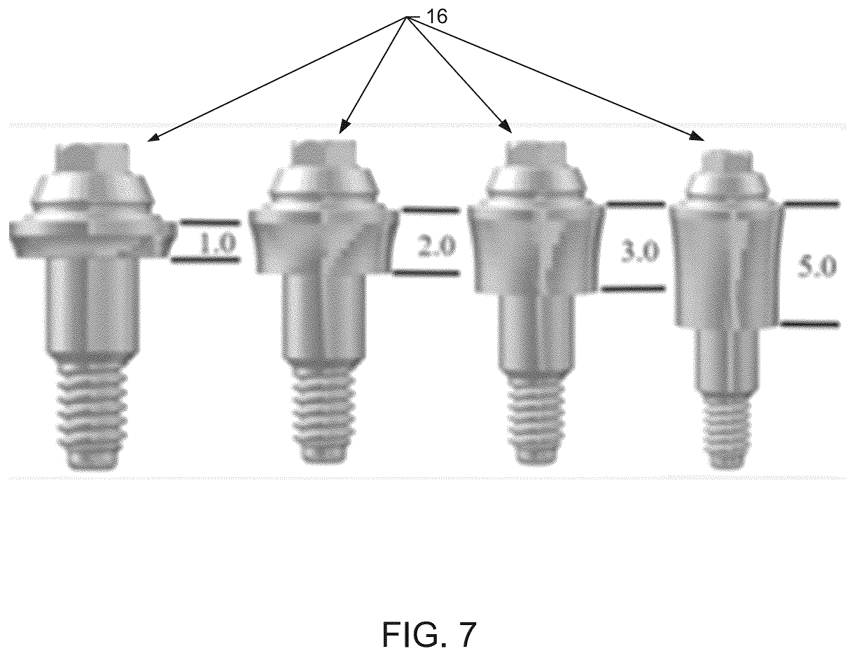

FIG. 7 may provide a non-limiting example of abutments 16 of different shapes, sizes, and dimensions that may have a male thread at a base thereof. In one embodiment, the interior bore of each pillar 12 may have a female thread which may be configured to receive one (or more) abutment bases. Responsive to the base of each abutment 16 being secured within the interior bore of a corresponding pillar 12, the abutment 16 may receive an artificial dental prosthesis. Thus, the dental prosthesis may ultimately be operably coupled to implant 10 via abutment bases and abutments 16.

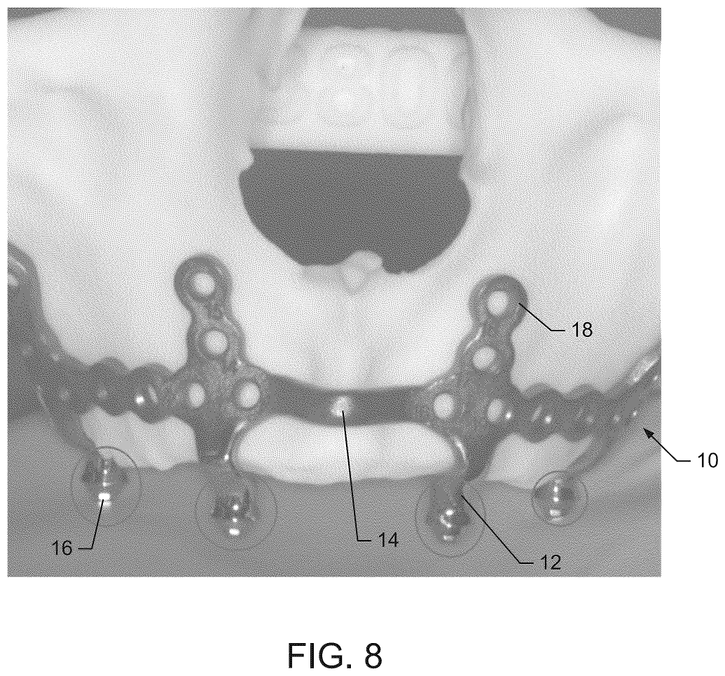

In some cases, such as the embodiment depicted in FIG. 8, each pillar 12 may be treated with a polishing treatment. The polishing treatment may remove a small amount of material from each pillar 12 to reduce the surface roughness of each pillar 12. In some cases, the abutments 16 may also be treated with the polishing treatment. In this regard, the pillars 12 and the abutments 16 may be the portions of the implant 10 that interact with, and come into direct contact with, the soft tissues of the patient. As such, treating the pillars 12 and the abutments 16 with the polishing treatment may prevent bacteria and plaque buildup on these components that may contact the patient's soft tissues, which may be desirable. On the other hand, the support structure 14 may remain un-treated by the polishing treatment. As such, the surface of the material at the support structure 14 may remain rough and gritty, which may promote osseointegration between the patient and the implant 10.

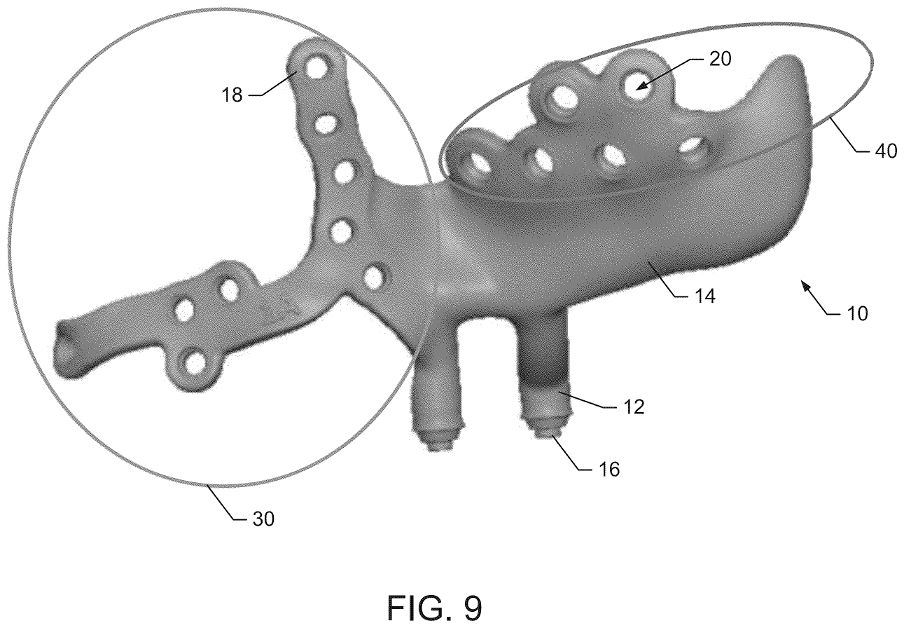

FIG. 9 depicts a perspective view of a portion of the support structure 14 in accordance with an example embodiment. In FIG. 9, the support structure 14 may be divided into a first region 30 and a second region 40. The first and second regions (30, 40) may be operably coupleable to different parts of the patient's bone. As such, the first and second regions (30, 40) of the support structure 14 may therefore also employ different types and different sizes of fastening members 15. For example, in some cases, surgeons may prefer to use a combination of locking fastening members and non-locking fastening members to operably couple the implant 10 to the patient's bone. In this regard, the locking fastening members may promote load-sharing as opposed to load bearing, and the non-locking fastening members may help seat the implant better on the bone. In the embodiment shown in FIG. 9, the locking fastening members may be disposed in the second region 40 and the non-locking fastening members may be disposed in the first region 30. However, in some example embodiments, the respective locations of the locking and non-locking fastening members may vary on a case-by-case basis.

In some other cases, the first and second regions (30, 40) of the support structure 14 may also employ different sizes of fastening members. In this regard, depending on the size of the bone to which the fastening member is operably coupled to, the fastening members may have different diameters. For example, in some cases, surgeons may prefer to use small fixation fastening members (approximately 1.5 mm diameter) at the piriform aperture, as well as larger fastening members (approximately 2.0-2.3 mm diameter) in larger bones (e.g. zygoma) to add increased strength and stability to the operable coupling of the support structure 14 to the bone. In the embodiment shown in FIG. 9, the larger fastening members may be disposed in the second region 40 and the small fixation fastening members may be disposed in the first region 30. However, in some example embodiments, the respective locations of the small and large fastening members may vary on a case by case basis.

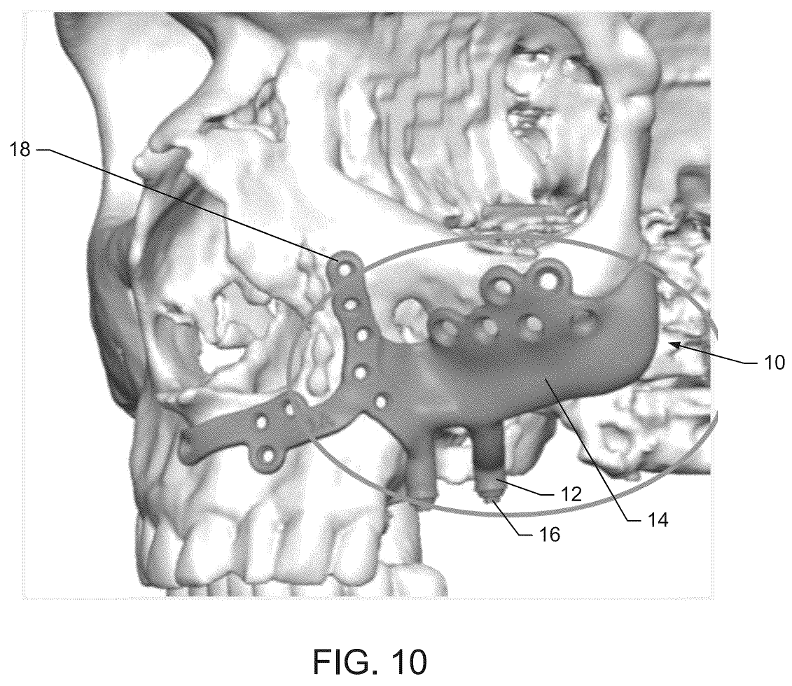

FIG. 10 illustrates a left side view of the support structure 14 in accordance with an example embodiment. In some cases, the support structure 14 may have a material thickness of between approximately 1.2 mm and approximately 10 mm. A thicker implant may be necessary in cases where restoring facial symmetry due to massive bone loss may be desired, such as the embodiment shown in FIG. 10.

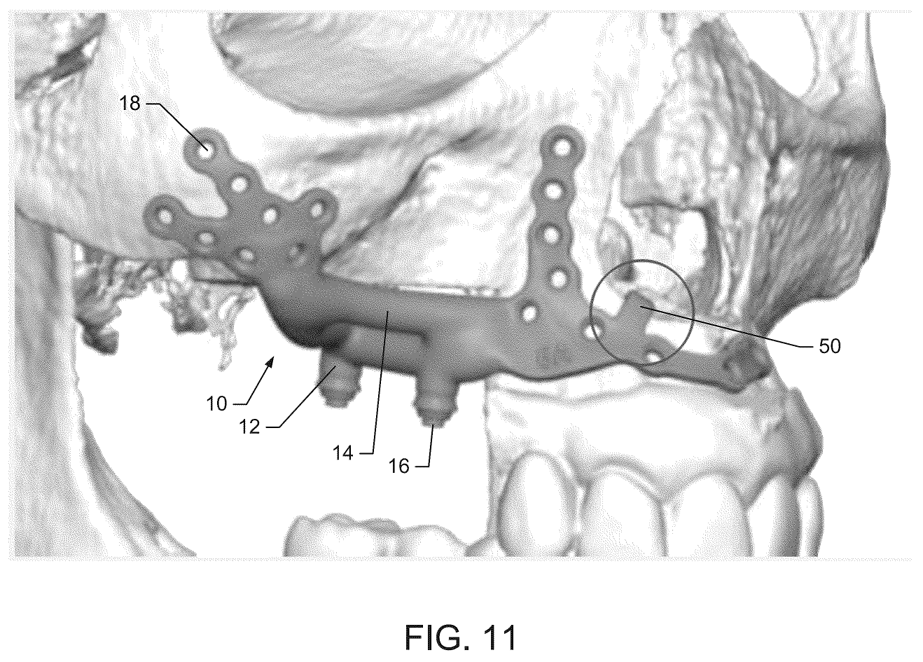

FIG. 11 depicts a perspective view of the support structure 14 according to an example embodiment. In the embodiment of FIG. 11, the implant 10 may include one or more registration tabs 50 formed at the support structure 14. The registration tabs 50 may extend away from the support structure 14 and interface with the bone of the patient. In this regard, the registration tabs 50 may engage or interface with specific bone structures or anatomical features of the patient to aid the implant in finding its designed/intended location within the patient.

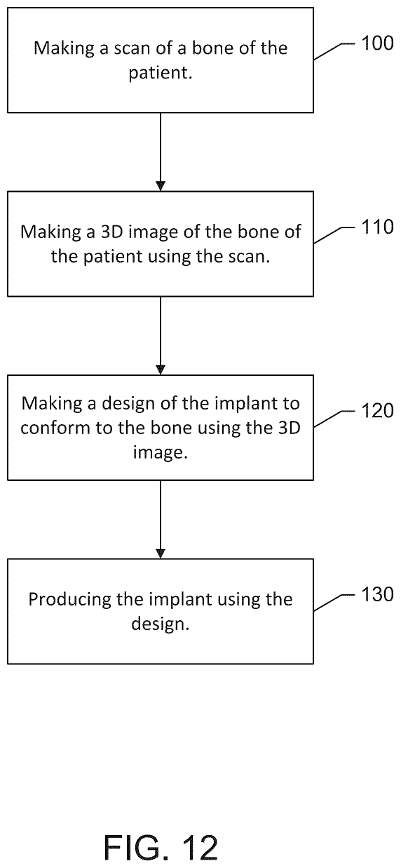

FIG. 12 illustrates a flow chart of a method of employing an implant 10 on a patient in accordance with an example embodiment. The method may include making a scan of a bone of the patient at operation 100 and making a 3D image of the bone of the patient using the scan at operation 110. The method may further include making a design of the implant 10 to conform to the bone using the 3D image at operation 120 and producing the implant 10 using the design at operation 130. In some cases, the method may further include operably coupling the implant 10 to the patient, and operably coupling the prosthesis to the implant 10. In an example embodiment, the prosthesis may be operably coupled to the implant via the abutments 16 disposed at the pillars 12 of the implant 10.

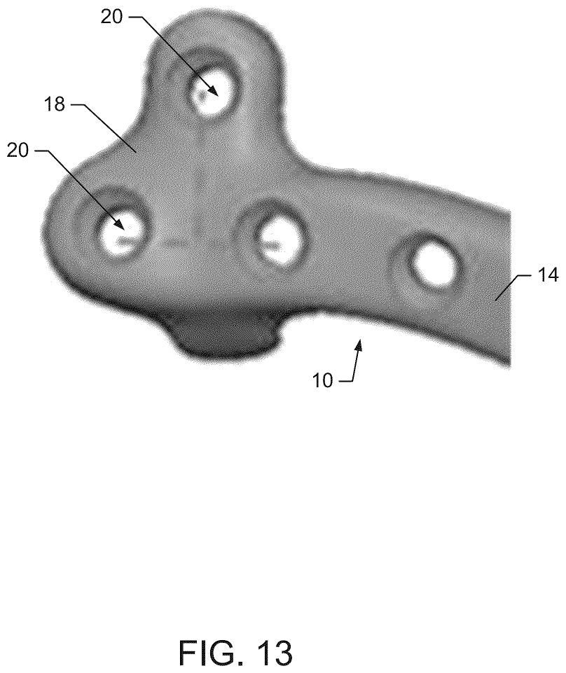

FIG. 13 depicts a close-up perspective view of the securing portion 18 according to an example embodiment. In the embodiment of FIG. 13, the securing portion 18 of the implant 10 may include one or more additional rows of annular portions 20 which may be configured to receive additional fastening members 15 therein. In this regard, the securing portions 18 may include the additional rows of annular portions 20 at locations where the implant 10 may be more difficult to operably couple to the patient either due to anatomical structures or the geometry of the implant 10. In some cases, the additional rows of annular portions 20 may include at least one annular portion 20. In an example embodiment, the additional rows of annular portions 20 may include at least one additional row of annular portions 20, but in some other cases more than one additional row may also be added. In some cases, the one or more additional rows of annular portions 20 may extend substantially parallel to a first row of annular portions 20.

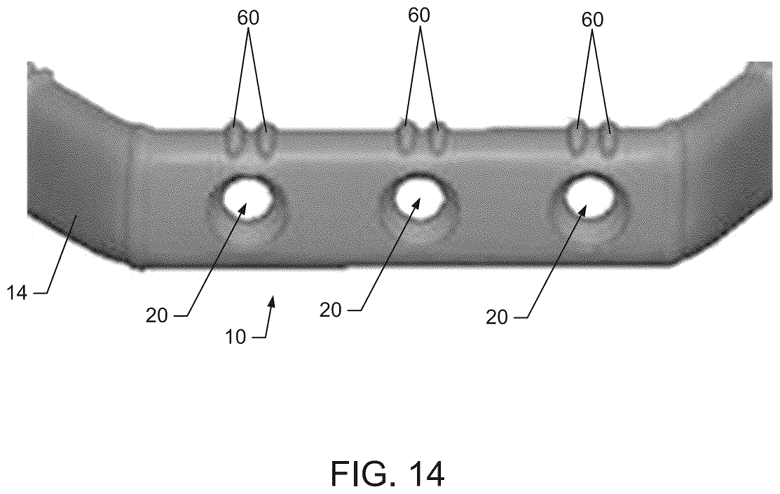

FIG. 14 depicts a close-up view of the support structure 14 according to an example embodiment. In the embodiment of FIG. 14, the implant 10 may include one or more intraoral hole finders 60 which may be formed at the support structure 14. The intraoral hole finders 60 may be protrusions disposed on a side of the support structure 14, and each intraoral hole finder 60 may indicate the location of a corresponding annular portion 20 in the support structure 14. In this regard, the intraoral hole finders 60 may be aligned with corresponding annular portions 20 in the support structure 14. In some cases, a plurality of intraoral hole finders 60 may be disposed proximate to each annular portion 20. In the particular example depicted in FIG. 14, each annular portion 20 may include two corresponding intraoral hole finders 60 disposed a distance apart from each other to define a gap therebetween the gap of some example embodiments may align with a center of the annular portion 20 to which the intraoral hole finders 60 may be disposed proximate. In some cases, the implant may use intraoral hole finders 60 from a locking screw part set, and the position of the intraoral hole finders 60 may match a direction of the screwdriver/surgical tool approach.

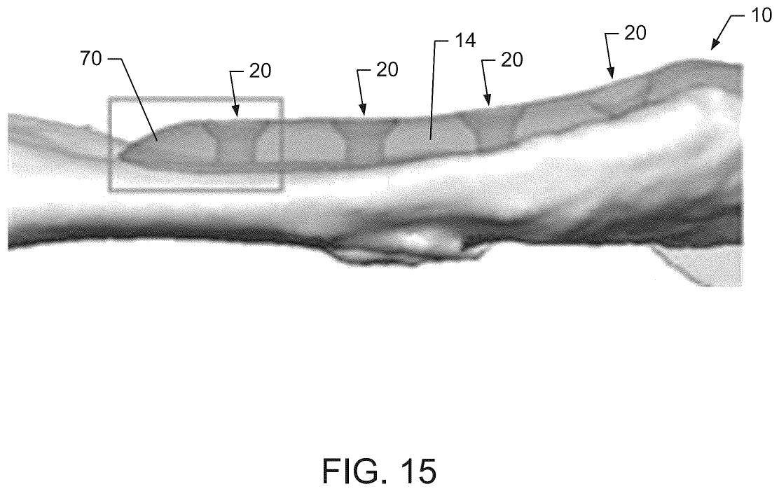

FIG. 15 depicts a close-up bottom view of the support structure 14 according to an example embodiment. In the embodiment of FIG. 15, the support structure 14 may include a tapered end 70. The tapered end 70 may taper towards the bone of the patient to provide a smoother transition between bone and implant 10, and to reduce the harshness of the implant 10 within the patient. In some cases, the tapered end 70 of the support structure 14 may improve the healing experience of the patient by reducing the severity of the angles and edges of the implant 10 to more closely mimic natural anatomical structures. At the tapered end 70, the thickness of the support structure 14, which may define a distance from the bone of the patient to an outermost surface of the support structure 14, may gradually be reduced over a length of the tapered end 70. In FIG. 15, the support structure 14 may be shown transparently to help visualize the shape of the support structure 14 and the operable coupling of the support structure 14 to the bone.

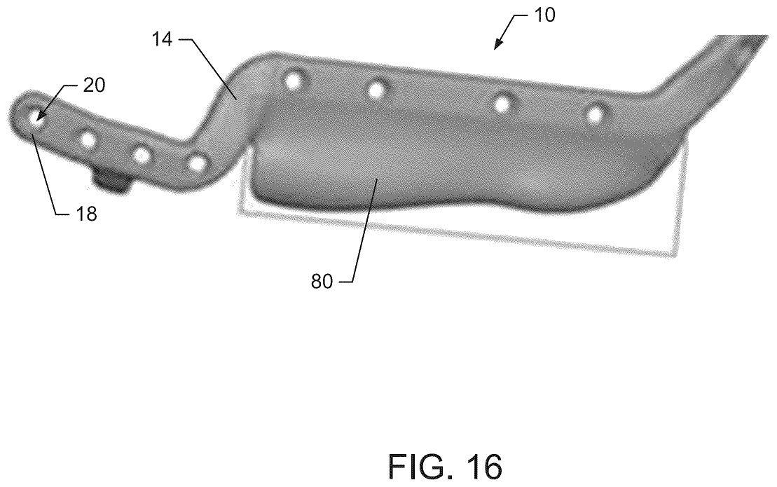

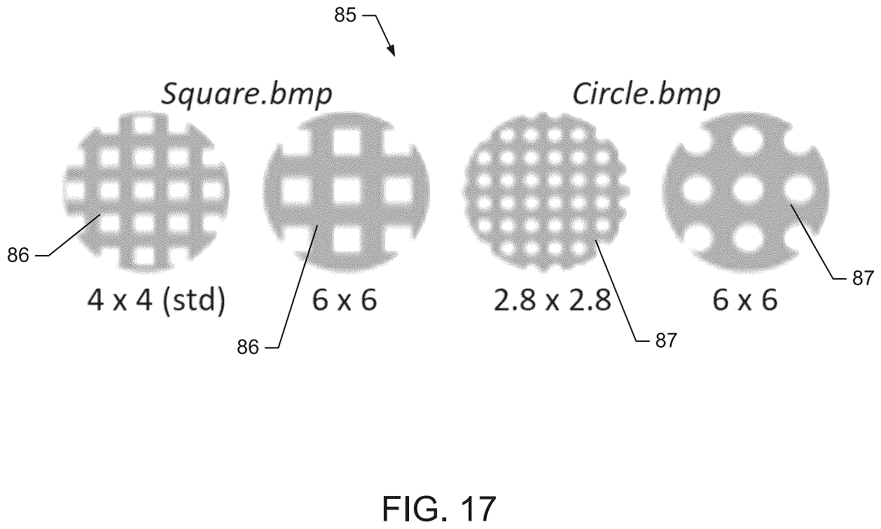

FIG. 16 depicts a close-up perspective view of the support structure 14 according to an example embodiment. In the embodiment of FIG. 16, the support structure 14 may include a crib extension 80, or in other words, an endoprosthesis. The crib extension 80 may extend away from the support structure 14 and interface with the bone of the patient. In this regard, the crib extension 80 may engage or interface with specific bone structures or anatomical features of the patient to augment the implant in cases where the patient may have a bony defect. In this regard, in some cases, the crib extension 80 may be non-load bearing. Instead, the crib extension 80 may be disposed at a portion of the support structure 14 proximate to where the patient may have the bony defect and may provide additional protection and support for this portion of the patient. In an example embodiment, the crib extension 80 may not include any annular portions 20 for receiving any fastener members 15 therein. In some cases, the crib extension 80 may be monolithically formed with the support structure 14, and thus may be formed from the same material as the support structure 14. In an example embodiment, the crib extension 80 may have a material thickness of approximately 1 mm. In some cases, the material of the crib extension 80 may be completely solid, but in some other cases, the material of the crib extension 80 may be produced in a 2D mesh pattern 85. The 2D mesh pattern 85 will be described in further detail below in reference to FIG. 17.

FIG. 17 depicts a close up view of the 2D mesh pattern 85 of the crib extension 80 according to an example embodiment. In the embodiment of FIG. 17, the 2D mesh pattern 85 may include various different sizes and shapes of material. Which size and shape is used may depend on factors such as the surgeon's preference, the particular use case, and the patient. In some cases, the 2D mesh pattern 85 may include a square pattern 86. In this regard, the square pattern 86 may be disposed in a 4×4 layout, or in a 6×6 layout. In some cases, the 4×4 layout may include smaller squares than the 6×6 layout. In an example embodiment, the 4×4 layout may be the standard option for the square pattern 86 2D mesh 85. In some other cases, the 2D mesh pattern 85 may include a circle pattern 87. In this regard, the circle pattern 87 may be disposed in a 2.8×2.8 layout, or in a 6×6 layout. In some cases, the 2.8×2.8 layout may include smaller circles than the 6×6 layout. In an example embodiment, the 2.8×2.8 layout of the circle pattern 87 may be the recommended choice for the 2D mesh pattern 85 of the crib extension 80. In some cases, the 2D mesh pattern 85 may include at least approximately 2 mm wide of material along the border of the 2D mesh pattern 85.

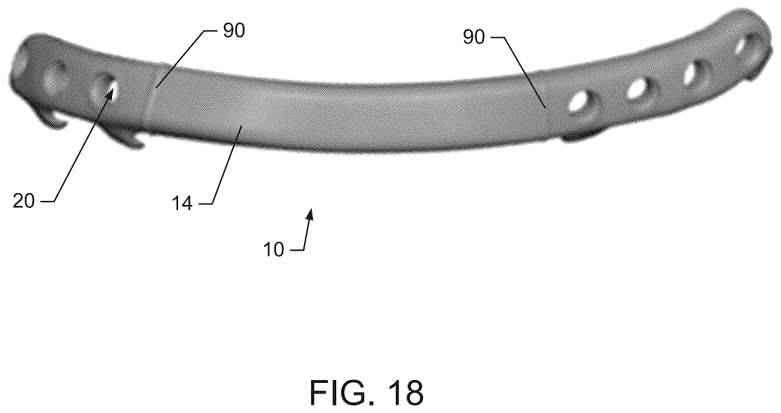

FIG. 18 depicts a close-up view of the support structure 14 according to an example embodiment. In the embodiment of FIG. 18, the implant 10 may include one or more anatomy markings 90 which may be formed at the support structure 14. Similar to the intraoral hole finders 60, the anatomy markings 90 may be protrusions disposed on a side of the support structure 14. Each anatomy marking 90 may indicate the location of a corresponding anatomical structure of the patient. In some cases, each anatomy marking 90 may extend across an entire width of the support structure 14. In an example embodiment, the anatomy markings 90 may be 0.3 mm thick by 1 mm wide circle ridges that may extend entirely around the support structure 14. In this regard, the anatomy markings 90 may add additional thickness to the support structure 14 of approximately 0.15 mm per side of the support structure 14.

In an example embodiment, an implant operably coupleable to a bone of a patient may be provided. The implant may include at least one securing portion which may be configured to receive fastening members to operably couple the implant to the bone, a support structure which may be conformal to contours of the bone, a plurality of pillars which may extend from, and be formed integrally with, the support structure, and a plurality of abutments which may be disposed at the plurality of pillars. The plurality of abutments may be operably coupleable to a prosthesis.

In some embodiments, the features of the implant described above may be augmented or modified, or additional features may be added. These augmentations, modifications and additions may be optional and may be provided in any combination. Thus, although some example modifications, augmentations and additions are listed below, it should be appreciated that any of the modifications, augmentations and additions could be implemented individually or in combination with one or more, or even all of the other modifications, augmentations and additions that are listed. As such, for example, the plurality of abutments may be removably operably coupled to the plurality of pillars. In an example embodiment, each of the plurality of pillars may include a receiving portion which may be configured to receive at least one abutment of the plurality of abutments. In some cases, each of the plurality of abutments may differ in size. In an example embodiment, each of the plurality of abutments may be pre-milled into corresponding ones of the plurality of pillars. In some cases, each of the plurality of abutments may be formed integrally with the plurality of pillars of the support structure. In an example embodiment, at least some of the plurality of pillars may have a different height to adjust to a bone structure of the patient. In some cases, each of the plurality of pillars may be angulated relative to an occlusal plane of the patient. In an example embodiment, the plurality of pillars may be treated with a polishing treatment and the support structure may remain un-treated. In some cases, the plurality of abutments may be treated with a polishing treatment. In an example embodiment, the fastening members may include locking fastening members and non-locking fastening members. In some cases, the implant may be operably coupled to the bone of the patient via both the locking fastening members and the non-locking fastening members. In an example embodiment, the locking fastening members may be disposed at a first region of the support structure and the non-locking fastening members may be disposed at a second region of the support structure. In some cases, the first region is different from the second region. In an example embodiment, the fastening members may include various different diameters. In some cases, a diameter of a particular fastening member may be based on a size of the bone to which the particular fastening member may be operably coupled. In an example embodiment, the diameter of the particular fastening member may be between approximately 1.5 mm and approximately 2.3 mm. In some cases, the support structure may have a material thickness of between approximately 1.2 mm and approximately 10 mm. In an example embodiment, the implant may further comprise one or more registration tabs formed at the support structure. In some cases, the registration tabs may extend away from the support structure and may interface with the bone of the patient.

Some example embodiments may provide for method of employing an implant on a patient. The method may include the steps of making a scan of a bone of the patient, making a 3D image of the bone of the patient using the scan, making a design of the implant to conform to the bone using the 3D image, and producing the implant using the design. The method may optionally further include the steps of operably coupling the implant to the patient, and operably coupling the prosthesis to the implant. The prosthesis may be operably coupled to the implant via a plurality of abutments disposed at a plurality of pillars of the implant.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe exemplary embodiments in the context of certain exemplary combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. In cases where advantages, benefits or solutions to problems are described herein, it should be appreciated that such advantages, benefits and/or solutions may be applicable to some example embodiments, but not necessarily all example embodiments. Thus, any advantages, benefits or solutions described herein should not be thought of as being critical, required, or essential to all embodiments or to that which is claimed herein. Although specific terms are employed herein, they are used in a generic and descriptive sense only and not for purposes of limitation.

What is claimed:

1. An implant operably coupleable to a bone of a patient, the implant comprising:
at least one securing portion configured to receive fastening members to operably couple the implant to the bone;
a support structure conformal to contours of the bone;
a plurality of pillars extending from, and formed integrally with, the support structure; and
a plurality of abutments disposed at the plurality of pillars, the plurality of abutments being operably coupleable to a prosthesis.

2. The implant of claim 1, wherein the plurality of abutments are removably operably coupled to the plurality of pillars.

3. The implant of claim 2, wherein each of the plurality of pillars comprises a receiving portion configured to receive at least one abutment of the plurality of abutments.

4. The implant of claim 3, wherein each of the plurality of abutments differs in size.

5. The implant of claim 1, wherein each of the plurality of abutments are pre-milled into corresponding ones of the plurality of pillars, and wherein each of the plurality of abutments are formed integrally with the plurality of pillars of the support structure.

6. The implant of claim 1, wherein at least some of the plurality of pillars have a different height to adjust to a bone structure of the patient.

7. The implant of claim 1, wherein each of the plurality of pillars is angulated relative to an occlusal plane of the patient.

8. The implant of claim 1, wherein the plurality of pillars are treated with an electropolishing treatment and the support structure remains un-treated.

9. The implant of claim 1, wherein the plurality of abutments are treated with an electropolishing treatment.

10. The implant of claim 1, wherein the fastening members comprise locking fastening members and non-locking fastening members, and
wherein the implant is operably coupled to the bone of the patient via both the locking fastening members and the non-locking fastening members.

11. The implant of claim 10, wherein the locking fastening members are disposed at a first region of the support structure and the non-locking fastening members are disposed at a second region of the support structure, and
wherein the first region is different from the second region.

12. The implant of claim 1, wherein the fastening members comprise various different diameters, and
wherein a diameter of a particular fastening member is based on a size of the bone to which the particular fastening member is operably coupled.

13. The implant of claim 12, wherein the diameter of the particular fastening member is between approximately 1.5 mm and approximately 2.3 mm.

14. The implant of claim 1, wherein the support structure has a material thickness of between approximately 1.2 mm and approximately 10 mm.

15. The implant of claim 1, wherein the implant further comprises one or more registration tabs formed at the support structure, and
wherein the registration tabs extend away from the support structure and interface with the bone of the patient.

16. A method of employing an implant on a patient, the method comprising:
making a scan of a bone of the patient;
making a 3D image of the bone of the patient using the scan;
making a design of the implant to conform to the bone using the 3D image; and
producing the implant using the design.

17. The method of claim 16, further comprising:
operably coupling the implant to the patient; and
operably coupling a prosthesis to the implant,
wherein the prosthesis is operably coupled to the implant via a plurality of abutments disposed at a plurality of pillars of the implant.

18. The method of claim 16, wherein the implant is operably coupled to the patient via fastening members,
wherein the fastening members comprise locking fastening members and non-locking fastening members, and
wherein the implant is operably coupled to the bone of the patient via both the locking fastening members and the non-locking fastening members.

19. The method of claim 18, wherein the fastening members comprise various diameters between approximately 1.5 mm and approximately 2.3 mm.

20. The method of claim 16, wherein producing the implant using the design further comprises treating a plurality of pillars with an electropolishing treatment and leaving a support structure un-treated.
